(19) United States
(12) Patent Application Publication
(10) Pub. No.: US 2024/0132443 A1
(43) Pub. Date: Apr. 25, 2024

(54) METHOD FOR CHLORINATING BENZALDEHYDE OXIMES

(71) Applicant: BAYER AKTIENGESELLSCHAFT, Leverkusen (DE)

(72) Inventors: Anton LISHCHYNSKYI, Leverkusen (DE); Frank MEMMEL, Leverkusen (DE); Mark James FORD, Leverkusen (DE); Stefan MOCZARSKI, Leverkusen (DE); Heiko SCHIRMER, Leverkusen (DE)

(21) Appl. No.: 18/554,920

(22) PCT Filed: Apr. 8, 2022
(86) PCT No.: PCT/EP2022/059428

§ 371 Date: Oct. 11, 2023

(30) Foreign Application Priority Data
Apr. 13, 2021 (EP) 21168079.8

(51) Int. Cl. C07C 249/12

(52) U.S. Cl. CPC C07C 249/12

(57) ABSTRACT

The present invention relates to a novel process for preparing chlorobenzaldehyde oximes of the general formula (I).

The present invention relates to a novel process for preparing chlorobenzaldehyde oximes of the general formula (I).

Chlorobenzaldehyde oximes of the general formula (I) are important precursors of active agrochemical ingredients (cf. WO 2018/228985) and active pharmaceutical ingredients (e.g. DNA-binding agents: Woods, Craig R. et al. Bioorganic & Medicinal Chemistry Letters, 12 (18), 2647-2650; 2002).

Numerous chlorination methods are described in the prior art; for example, WO 2004/29066 teaches the preparation of chlorobenzaldehyde oximes by the reaction of oximes with N-chlorosuccinimide (NCS) and subsequent aqueous work-up (extraction with EtOAc/H₂O). However, only small amounts (2.45 g) of the chlorobenzaldehyde oximes obtained were isolated in solid form in the process described. In principle, the isolation of chlorobenzaldehyde oximes in solid form on an industrial scale is undesirable, however, since chlorobenzaldehyde oximes are often high-energy compounds which exhibit a high tendency to decompose. The process described in WO 2004/29066 uses dimethylformamide (DMF) as solvent. It is known, however, that the use thereof as solvent on an industrial scale may be problematic. This is due to the strongly exothermic reaction between DMF and the chlorinating agent, which then possibly proceeds in an uncontrolled manner. (OPRD 2020, 24, 1586; Bull. Chem. Soc. Jpn. 1994, 67, 156).

The Journal of Enzyme Inhibition and Medicinal Chemistry; vol. 31; issue 6; (2016); pp. 964-973 teaches the chlorination of oximes using trichloroisocyanuric acid (TCCA) with triethylamine as base; DMF is not used as solvent in this case, however it was observed that the chloroximes tend to degrade in a basic environment by formation of the nitrile oxides, which may lead to losses in yield (e.g. dimerization of the nitrile oxides to form furoxans: “Kinetics and Mechanism of 1,3-Dipolar Cycloadditions” by Prof. Dr. R. Huisgen, Angew. Chem. 1963, 75, 742-754, page 751; “Fragmentation of Nitrile Oxides with Triethylamine” Tetrahedron Lett. 1983, 24, 4377-4380).

The invention was therefore based on the object of providing a process for chlorinating benzaldehyde oximes which, on the one hand, can dispense with DMF as solvent and, on the other hand, does not bring about the losses in yield caused by relatively strong bases, such as triethylamine, and thus simultaneously is cost-effective and can be used on an industrial scale.

The object was achieved according to the invention by a process for preparing chlorobenzaldehyde oximes of the general formula (I)

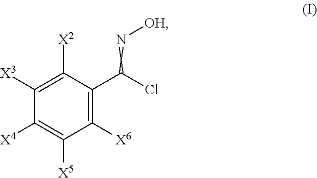

in which
X² is H, C₁-C₄ alkyl, C₁-C₄ fluoroalkyl, C₁-C₄ fluoroalkoxy, C₁-C₄ alkoxy, fluorine, CN,
X³ is H, C₁-C₄ alkyl, C₁-C₄ fluoroalkyl, C₁-C₄ fluoroalkoxy, C₁-C₄ alkoxy, fluorine, chlorine, CN,
X⁴ is H, C₁-C₄ alkyl, C₁-C₄ fluoroalkyl, C₁-C₄ fluoroalkoxy, C₁-C₄ alkoxy, fluorine, CN,
X⁵ is H, C₁-C₄ alkyl, C₁-C₄ fluoroalkyl, C₁-C₄ fluoroalkoxy, C₁-C₄ alkoxy, fluorine, chlorine, CN,
X⁶ is H, C₁-C₄ alkyl, C₁-C₄ fluoroalkyl, C₁-C₄ fluoroalkoxy, C₁-C₄ alkoxy, fluorine, CN,
characterized in that the compounds of the general formula (II)

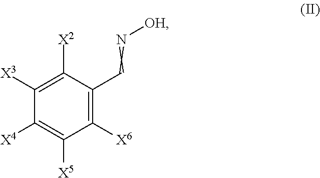

in which
X² to X⁶ have the meanings stated above,
are converted into compounds of the general formula (I) with the aid of trichloroisocyanuric acid (TCCA) and an amide base.

Preferred definitions of the radicals for the compounds of the general formulae (I) and (II) are as follows:
X² is H, methyl, trifluoromethyl, difluoromethyl, difluoromethoxy, trifluoromethoxy, fluorine, methoxy, CN,
X³ is H, methyl, trifluoromethyl, difluoromethyl, difluoromethoxy, trifluoromethoxy, fluorine, chlorine, methoxy, CN,
X⁴ is H, methyl, trifluoromethyl, difluoromethyl, difluoromethoxy, trifluoromethoxy, fluorine, methoxy, CN,
X⁵ is H, methyl, trifluoromethyl, difluoromethyl, difluoromethoxy, trifluoromethoxy, fluorine, chlorine, methoxy, CN,
X⁶ is H, methyl, trifluoromethyl, difluoromethyl, difluoromethoxy, trifluoromethoxy, fluorine, methoxy, CN.

Particularly preferred definitions of the radicals for the compounds of the general formulae (I) and (II) are as follows:
X² is H,
X³ is H, methyl, trifluoromethyl, difluoromethyl, fluorine, chlorine, methoxy, CN,
X⁴ is fluorine, H,
X⁵ is H, methyl, trifluoromethyl, difluoromethyl, fluorine, chlorine, methoxy, CN,
X⁶ is H.

Very particularly preferred definitions of the radicals for the compounds of the general formulae (I), (II) are as follows:
X² is H,
X³ is H, fluorine,
X⁴ is H, fluorine,
X⁵ is H, fluorine,
X⁶ is H.

Most preferred definitions of the radicals for the compounds of the general formulae (I) and (II) are as follows:
X² is H,
X³ is fluorine,
X⁴ is H,
X⁵ is fluorine,
X⁶ is H.

The compounds of the formula (I) may be present as mixtures of geometric isomers:

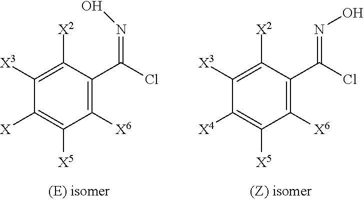

The ratio between E and Z isomers varies.

### Elucidation of the Processes and Intermediates

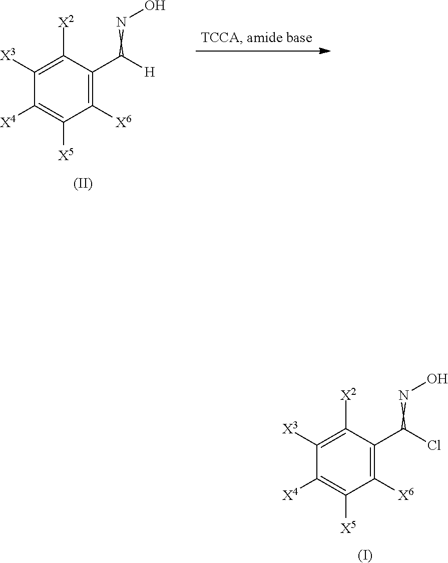

Process for preparing chlorobenzaldehyde oximes of the formula (I), characterized in that the compounds of the general formula (II) are converted into compounds of the general formula (I) with the aid of trichloroisocyanuric acid (TCCA) and an amide base.

The process according to the invention has the advantage of avoiding DMF as solvent. The risk of the reaction proceeding in a strongly exothermic and uncontrolled manner is thereby minimized The reaction is therefore suitable for performance on a large scale.

Further suitable amide bases are, for example, dibutylformamide (DBF), diethylformamide (DEF), or dimethylacetamide (DMAc), with dibutylformamide being preferred.

In the process according to the invention, preferably 0.5-2 equivalents of amide base are used, based on the benzaldehyde oxime (II), particularly preferably 1-1.5 equivalents. Preferably 0.3-0.4 equivalents of TCCA are used based on the benzaldehyde oxime (II) (0.9-1.3 equivalents of “Cl”).

Furthermore, the reaction mixture can be worked up free from water and the precipitated cyanuric acid can be removed by filtration.

The chlorination is usually performed in a temperature range from −10° C. to 40° C., preferably −5° C. to 10° C., particularly preferably 0 to 5° C.

The chlorination is furthermore performed in the presence of a solvent or diluent, preferred solvents being tetrahydrofuran, Me-THF, acetonitrile, N,N-dimethylacetamide, toluene, ethyl acetate, isopropyl acetate, methyl-tert-butyl ether.

The trichloroisocyanuric acid (TCCA) is added to the benzaldehyde oxime of the formula (II) in solid form or as a freshly prepared solution in ethyl acetate, isopropyl acetate or acetonitrile. The concentration of the solution depends here on the solubility of TCCA in the respective solvent. For example, up to approx. 25 w/w % dissolve in ethyl acetate and up to approx. 20 w/w % dissolve in isopropyl ester.

### EXAMPLES

The present invention is elucidated in more detail by the examples that follow, without restriction of the invention thereto.

### Measurement Methods

The products were characterized by ¹H and/or ¹⁹F NMR spectroscopy and/or HPLC and/or LC-MS (Liquid Chromatography Mass Spectrometry).

The NMR spectra were determined using a Bruker Avance 400 fitted with a flow probe head (volume 60 μl). In individual cases, the NMR spectra were measured with a Bruker Avance II 600.

### Example 1 (Addition of TCCA in Solid Form)

313.50 g of an N-(3,5-difluorobenzylidene)hydroxylamine solution (31.9 w/w % in toluene/THF) were initially charged into a 2 l four-necked flask with precision glass stirrer and dropping funnel, under a protective argon gas atmosphere, at 23° C. 151.66 g of N,N-dibutylformamide were then added via the dropping funnel over the course of 15 min, with stirring. After the solution had been cooled to 0° C. in an ice bath, 50.06 g of TCCA were added in portions of in each case approx. 0.46 g by means of a solids metering system, with stirring (210 rpm) over the course of 2 h. The temperature during the addition was kept below 5° C. here. After the addition of TCCA had been concluded, stirring of the reaction mixture was continued for a further 30 minutes at 0° C. The HPLC analysis showed a proportion of 92.8% of 3,5-difluoro-N-hydroxybenzenecarboximidoyl chloride and no remaining N-(3,5-difluorobenzylidene)hydroxylamine. Subsequently, the reaction mixture was heated to 23° C. with stirring and stirring was continued for 1 h. The cyanuric acid formed was filtered off as a white solid and washed twice with 25 ml of toluene in each case, with 460.00 g of a 3,5-difluoro-N-hydroxybenzenecarboximidoyl chloride solution being obtained. The analysis by ¹⁹F Q-NMR gave a yield of 84% at a concentration of 22.4 w/w %. After drying in air, 26.09 g of cyanuric acid (95%) could also be recovered.

¹H NMR (401 MHz, CDCl₃): δ (ppm)=6.84-6.89 (m, 1H), 7.37-7.45 (m, 2H), 10.86 (bs, 1H).

¹⁹F NMR (377 MHz, CDCl₃): δ (ppm)=−109.3 (m, 2F).

### Example 2 (Addition of TCCA as a 20 wt % Solution in Isopropyl Acetate)

20.00 g of an N-(3,5-difluorobenzylidene)hydroxylamine solution (31.9 w/w % in toluene/THF) were initially charged into a 250 ml three-necked flask with magnetic stirrer and septum under a protective argon gas atmosphere at 23° C. and 9.68 g of N,N-dibutylformamide were added dropwise using a syringe over the course of 15 min with stirring. After the resulting solution had been cooled to 0° C. in an ice bath, 15.81 g of TCCA dissolved in isopropyl acetate (20 w/w %) were added to the reaction mixture over the course of 2 h using a syringe pump with continued stirring. The temperature was kept below 5° C. here. After the addition of TCCA had been completed, the reaction mixture was stirred for a further 30 minutes at 0° C., heated to 23° C. and stirred for a further hour, and then 7.81 g of fluorobenzene were added as internal standard (19F Q-NMR). The resulting reaction mixture showed, via HPLC, a complete conversion of the N-(3,5-difluorobenzylidene)hydroxylamine, with a yield of 87% (¹⁹F Q-NMR).

### Example 3 (19 kg Solution (19.7 w/w % in Toluene/THF) Industrial-Scale Batch)

19.2 kg of N-(3,5-difluorobenzylidene)hydroxylamine solution (19.7 w/w % in toluene/THF) were initially charged into a 50 l steel/enamel reactor under a protective nitrogen gas atmosphere and 5.7 kg of N,N-dibutylformamide were added at 15-20° C. After the resulting solution had been cooled to 0° C., 1.9 kg of TCCA dissolved in 10 l of isopropyl acetate (20 w/w %) were metered into the reaction mixture over the course of 90 min at 0-5° C., the mixture was stirred for a further 30 minutes at 0° C., the temperature of the mixture was adjusted to 20° C. and stirring was continued. The reaction solution was filtered off via a layer of kieselguhr and washed with 5 l of isopropyl acetate. The resulting product solution (33.7 kg) showed, via HPLC, a complete conversion of the N-(3,5-difluorobenzylidene)hydroxylamine, with a yield of 89% (19F Q-NMR).

### Example 4 (5 kg Industrial-Scale Batch)

5.0 kg of N-(3,5-difluorobenzylidene)hydroxylamine (94.0 w/w %) were initially charged into a 50 l steel/enamel reactor under a protective nitrogen gas atmosphere and dissolved in 3.8 l of toluene and 9.3 l of THF at 20° C., and 7.05 kg of N,N-dibutylformamide were added at 15-20° C. After the resulting solution had been cooled to 0° C., 2.5 kg of TCCA dissolved in 11.3 l of isopropyl acetate (20 w/w %) were metered into the reaction mixture over the course of 90 min at 0-5° C., the mixture was stirred for a further 30 minutes at 0° C., the temperature of the mixture was adjusted to 20° C. and stirring was continued. The reaction solution was filtered off via a layer of kieselguhr and washed with 2 l of isopropyl acetate. The resulting product solution (33.3 kg) showed, via HPLC, a complete conversion of the N-(3,5-difluorobenzylidene)hydroxylamine, with a yield of 86% (¹⁹F Q-NMR).

1. A process for preparing chlorobenzaldehyde oximes of general formula (I) 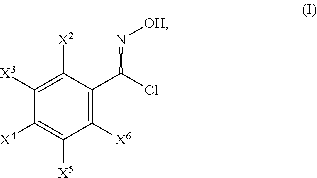
in which
X² is H, C₁-C₄ alkyl, C₁-C₄ fluoroalkyl, C₁-C₄ fluoroalkoxy, C₁-C₄ alkoxy, fluorine, or CN,
X³ is H, C₁-C₄ alkyl, C₁-C₄ fluoroalkyl, C₁-C₄ fluoroalkoxy, C₁-C₄ alkoxy, fluorine, chlorine, or CN,
X⁴ is H, C₁-C₄ alkyl, C₁-C₄ fluoroalkyl, C₁-C₄ fluoroalkoxy, C₁-C₄ alkoxy, fluorine, or CN,
X⁵ is H, C₁-C₄ alkyl, C₁-C₄ fluoroalkyl, C₁-C₄ fluoroalkoxy, C₁-C₄ alkoxy, fluorine, chlorine, or CN,
X⁶ is H, C₁-C₄ alkyl, C₁-C₄ fluoroalkyl, C₁-C₄ fluoroalkoxy, C₁-C₄ alkoxy, fluorine, or CN,
characterized in that benzaldehyde oxime compounds of general formula (II) 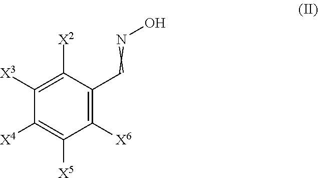
in which
X² to X⁶ have the meanings stated above,
are converted in a reaction into compounds of the general formula (I) in the presence of trichloroisocyanuric acid (TCCA) and an amide base.

2. The process according to claim 1, wherein the definitions of the radicals of the general formulae (I) and (II) are as follows:
X² is H, methyl, trifluoromethyl, difluoromethyl, difluoromethoxy, trifluoromethoxy, fluorine, methoxy, or CN,
X³ is H, methyl, trifluoromethyl, difluoromethyl, difluoromethoxy, trifluoromethoxy, fluorine, chlorine, methoxy, or CN,
X⁴ is H, methyl, trifluoromethyl, difluoromethyl, difluoromethoxy, trifluoromethoxy, fluorine, methoxy, or CN,
X⁵ is H, methyl, trifluoromethyl, difluoromethyl, difluoromethoxy, trifluoromethoxy, fluorine, chlorine, methoxy, or CN,
X⁶ is H, methyl, trifluoromethyl, difluoromethyl, difluoromethoxy, trifluoromethoxy, fluorine, methoxy, or CN.

3. The process according to claim 2, wherein the definitions of the radicals of the general formulae (I) and (II) are as follows:
X² is H,
X³ is H, methyl, trifluoromethyl, difluoromethyl, fluorine, chlorine, methoxy, or CN,
X⁴ is fluorine or H,
X⁵ is H, methyl, trifluoromethyl, difluoromethyl, fluorine, chlorine, methoxy, or CN,
X⁶ is H.

4. The process according to claim 3, wherein the definitions of the radicals of the general formulae (I) and (II) are as follows:
X² is H,
X³ is H or fluorine,
X⁴ is H or fluorine,
X⁵ is H or fluorine,
X⁶ is H.

5. The process according to claim 4, wherein the definitions of the radicals of the general formulae (I) and (II) are as follows:
X² is H,
X³ is fluorine,
X⁴ is H,
X⁵ is fluorine,
X⁶ is H.

6. The process according to claim 1, characterized in that the reaction is carried out at −10° C. to 40° C.

7. The process according to claim 6, characterized in that the reaction is carried out at −5° C. to 10° C.

8. The process according to claim 1, characterized in that 0.5-2 equivalents of amide base are used, based on the benzaldehyde oxime (II).

9. The process according to claim 1, characterized in that 0.3-0.4 equivalents of TCCA are used, based on the benzaldehyde oxime (II).

10. The process according to claim 1, characterized in that the amide base is dimethylformamide (DMF), dibutylformamide (DBF), diethylformamide (DEF) or dimethylacetamide (DMAc).

11. The process according to claim 10, characterized in that the amide base is dibutylformamide (DBF).
